(19) United States
(12) Patent Application Publication
(10) Pub. No.: US 2006/0058541 A1
(43) Pub. Date: Mar. 16, 2006

(54) Purifying polyoxyethylated castor oils with activated charcoal and pharmaceutical formulations thereof

(76) Inventors: Kai Zhang, Miami, FL (US); Gregory A. Smith, Miami, FL (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/182,041

(22) PCT Filed: Jan. 19, 2001
(86) PCT No.: PCT/US01/01749

§ 371 Date: Mar. 31, 2005

Related U.S. Application Data
(62) Division of application No. 60/177,459, filed on Jan. 20, 2000.

(51) Int. Cl. A61K 31/337; C07C 51/43

(52) U.S. Cl. 554/191; 514/449

(57) ABSTRACT

Disclosed are polyoxyethylated castor oils produced by preparing a suspension of activated charcoal and a polyoxyethylated castor oil; and separating the activated charcoal from the polyoxyethylated castor oil. The process removes impurities such as colorants and alkali metal cations. Also disclosed are compositions containing the castor oil and an active agent such as a pharmaceutical agent. The formulations have prolonged storage stability.

### TECHNICAL FIELD

The present invention relates to the removal of impurities from polyethoxylated castor oils, and formulations containing the treated castor oils and active agents.

### BACKGROUND OF THE INVENTION

Polyethoxylated castor oils are commonly used as solubilizing and/or dispersing agents for a variety of pharmaceutically active agents that are substantially insoluble in water. Impurities tend to catalyze the decomposition of the pharmaceutically active agents, particularly anti-neoplastic agents such as paclitaxel. In particular, it has been found that pharmaceutical compositions of paclitaxel in a co-solvent system containing dehydrated ethyl alcohol and commercial grade Cremophor EL® exhibit a loss of potency of greater than 60% after storage for 12 weeks at 50° C., the loss of which is attributable to the decomposition of paclitaxel during storage. In addition to the impurities such as colorants and dopants that are contained in the castor oil solution that is used to prepare the pharmaceutical composition, other impurities are formed during storage of the ultimate pharmaceutical formulation. Such impurities include fibrous precipitate of unknown composition, and which also cause loss of potency of the active agent.

U.S. Pat. No. 5,504,101 to Agharkar, et al., teaches a method for treating a polyoxyethylated castor oil with an acid or contacted with alumina to reduce carboxylate anion content, thereby extending shelf life and lower amounts of degradation by-products. U.S. Pat. No. 5,925,776 to Nikolayev, et al., teaches methods of reducing cation contents of polyoxyethylated castor oils by pre-treating the castor oil with a strong cationic exchange resin such as styrene divinyl benzene. International publication number WO 00/23070 to Ben Venue teaches a method for purifying polyoxyethylated castor oils by contacting a solution of castor oil and an alcohol with a column containing activated carbon, followed by contact with an ion exchange resin column to remove residual amounts of carbon.

### SUMMARY OF THE INVENTION

Applicants have devised a method for removing impurities from polyethoxylated castor oils without requiring cumbersome chromatographic procedures.

Hence, one aspect of the present invention is directed to a polyoxyethylated castor oil produced by the steps of preparing a suspension of activated charcoal in a polyoxyethylated castor oil (e.g., a commercial grade castor oil), and separating the activated charcoal from the polyoxyethylated castor oil. In preferred embodiments, the suspension is heated prior to separating and the separation of the activated charcoal from the castor oil is performed while the suspension is still heated. In a more preferred embodiment, impurities are removed by prepared by preparing a suspension of activated charcoal and a polyoxyethylated castor oil wherein the activated charcoal has a median surface area of about 900 m²/g, and is present in the suspension in an amount of from 3 to about 20% (w/w); heating said suspension at a temperature of from about 30° C. to about 60° C. for a period of time of from about 1 to about 6 hours; and filtering the heated suspension to separate the activated charcoal from the castor oil. The castor oils of the present invention have reduced impurities (e.g., colorants and alkali metal cations including K⁺ and Na⁺) relative to castor oils not having undergone the processes described herein.

Another aspect of the present invention is directed to the purification process per se, which entails preparing a suspension of activated charcoal in the polyoxyethylated castor oil under conditions of time and temperature so as to allow the impurities to be removed from the castor oil.

Another aspect of the present invention is directed to compositions and formulations containing a polyoxyethylated castor oil treated in accordance with the aforementioned process, and an active agent. The agent is soluble or dispersible in the castor oil. In preferred embodiments, the active agent is an anti-neoplastic agent, more preferably a taxane such as paclitaxel. More preferred compositions also contain, in addition to the paclitaxel, dehydrated ethanol and citric acid. Processes of making the compositions are also provided.

### BEST MODE OF CARRYING OUT THE INVENTION

Polyoxyethylated castor oils are produced by condensation of castor oil with ethylene oxide. Preferred oils are commercially available under the trade names Cremophor EL® and Cremophor EL-P (BASF). Other preferred castor oils include polyoxyl 35 castor oil and Cremphor RH60. Alternatively, Cremophor may be prepared in accordance with the methods disclosed in U.S. Pat. No. 3,070,499.

Activated charcoal is commercially available from several sources e.g., Calgon (Pittsburg, Pa.) and Spectrum Chemical Manufacturing Corp. (Gardena, Calif.). Alternatively, charcoal may be activated in accordance with standard procedures, notably by chemical treatment, or steam or some other heat source, charcoal can be “activated”. Bonhomme-Faivre, et al., Life Sci. 66(9):817-827 (2000), for example, discloses pinewood charcoal LSM (CECA-SA, 92 La Defense) and peat charcoal SX4 (Norit 93, Le Blanc Masnil), activated by steam and washed with phosphoric acid. The surface absorption for pinewood charcoal is 1,000 m²/g and 650 m²/g for peat charcoal. In general, the activated charcoal has a surface area ranging from about 500 to about 1300 m²/g (including sub-ranges thereof), and preferably a median surface area range of about 900 m²/g.

To reduce the impurities in a given polyoxyethylated castor oil, a predetermined amount of the oil is added together with activated charcoal to produce a suspension of the charcoal in the oil. The product is thus a free suspension of the activated charcoal, in contrast to the Ben Venue patent publication which entails contacting the castor oil with a column containing the charcoal. Persons skilled in the art will be able to determine the amounts of activated charcoal in order to remove impurities, in accordance with standard techniques. In general, the charcoal is present in the suspension in an amount of from 3 to about 20% (w/w), preferably from about 5 to about 10% (w/w), including sub-ranges thereof. The castor oil and activated charcoal are allowed to remain in contact under conditions (e.g., time and temperature) to allow removal or capture of impurities by the charcoal. Although not intending to be bound by any particular theory of operation, it is believed that the activated charcoal adsorbs the impurities. In preferred embodiments, the suspension is heated, generally at a temperature of from about 30° C. to about 60° C., and for a period of time from about 1 to about 6 hours, including sub-ranges thereof. It is believed that the heating enhances absorption of impurities and thus facilitates their removal from the castor oil. It is also preferred to stir the suspension, at least periodically. The activated charcoal is then separated from the castor oil. This separation is conveniently performed by at least one filtration step. In the event that two or more filtration steps are used, filters having successively smaller apertures are used. In preferred embodiments, the heated suspension is filtered while it is still heated. It is believed that the lesser viscosity of the heated suspension facilitates filtration. In less preferred embodiments, the heated suspension is allowed to cool to about room temperature, optionally with stirring. Other techniques for separating or removing activated charcoal are known in the art.

The treatment or purification process removes impurities from the castor oil. The main impurities removed include colorants (e.g., naturally occurring color-stuffs contained in castor oils, and dopants and other chemical additives that are included in the final commercial product) and alkali metal cations such as K⁺ and Na⁺. In preferred embodiments, the thus-treated castor oil has a color content of no greater than about 0.045 absorbance units (AU), measured at 425 nm/1 cm cell. In more preferred embodiments, the castor oil has a color content of no more than about 0.038 AU. With respect to cations, the castor oils of the present invention have a K⁺ content is no more than about 80 ppm, more preferably no more than about 50 ppm, and a Na⁺ content no more than about 10 ppm. In other preferred embodiments, the castor oil has a water content of no greater than about 2.5%.

The polyoxyethylated castor oil of the present invention disperse or solubilize a wide variety of active agents, including pharmaceutical agents e.g., anesthetics (e.g., benzocaine), immunosuppressive agents, anti-fungal agents (e.g., miconazole and clotrimazole), anti-bacterial agents (e.g., hexidine), non-steroidal anti-inflammatory agents (e.g., diclofenac), vitamins (e.g., A, D, E and K), cosmetics (e.g., deodorant and antiperspirant actives such as aluminum zirconium chloride, aluminum chloride and sodium bicarbonate) and feedstuff, and other agents such as excipients (e.g., solvents, thickeners, colors, dyes, flow aids, lubricants and non-volatile silicones such as cyclomethicone and clays e.g., bentonite. In preferred embodiments, the active agent is an anti-neoplastic agent. Suitable anti-neoplastic agents include teniposide, a semi-synthetic derivative of podophyllotoxin having the chemical name 4′-demethylepiodophyllotoxin 9-(4,6-O-2-thenylidene-β-D-glucopyranoside), camptothecin, a compound isolated from the stemwood of the Chinese tree, and taxanes (e.g., obtainable from the bark of Pacific yew trees). In more preferred embodiments, the anti-neoplastic agent is a taxane. Suitable taxanes include paclitaxel and its prodrugs (e.g., docetaxel), derivatives, pharmaceutically acceptable salts and metabolites thereof. Docetaxel (N-debenzoyl-N-tert-butoxycarbonyl-10-deacetyl paclitaxel) is commercially available under the trade name TAXOTERE® (Rhone-Poulenc-Rohrer S.A.). Taxol analogs and derivatives are disclosed in the literature, e.g., U.S. Pat. Nos. 6,103,698 and 6,136,990; WO 94/03093; Kingston, DGI. Taxol: the chemistry and structure-activity relationships of a novel anticancer agent. Trends Biotechnology 12: 222-227 (1994); and Alder, J D et al. Preclinical in vivo efficacy of two dihydrotaxane analogues against human and murine tumors. Br. J. Cancer 73:560-564 (1996). Other than docetaxel, other prodrugs include the 2′-onium salts of paclitaxel and docetaxel, particularly the 2′-methylpyridiniuin mesylate (2′-MPM) salts. Preferred metabolites of paclitaxel, designated A, B and C, are represented by the following formula:

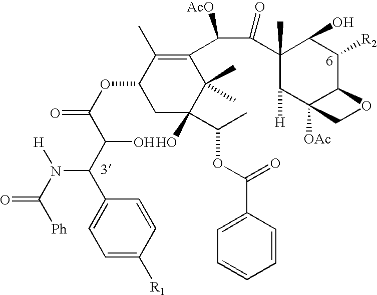

In the formula, metabolite A: R₁═H and R₂═OH; metabolite B: R₁═OH and R₂═H; and metabolite C: R₁═H and R₂═H.

In the case of taxanes, it is particularly preferred that the castor oil is contained in admixture with an alcohol such as dehydrated ethanol. A 50:50 mixture (w/w) is preferred. Stabilizers may also be added. Citric acid is a preferred stabilizer.

The products and methods of the present invention are further illustrated by the following example. The presentation of this example is by no way intended to limit applicants' invention in any way. Unless otherwise specified, all percentages are by weight.

### EXAMPLE

One hundred grams of charcoal were placed in an oven at 120° C. for about 16 hours. Nine hundred grams Cremophor EL® were added to make a 10% charcoal (w/w) Cremophor EL® slurry solution. The solution was heated to 45° C. with a hot plate for three hours with mechanical stirring, followed by cooling to room temperature, followed by stirring for two additional hours. The slurry solution was then filtered twice using a 10 μm filter and a 0.45 μm filter.

The paclitaxel formulation was prepared by dissolving 6 mg/ml of paclitaxel in 50/50 (v/v) commercial or pre-treated Cremophor EL® and dehydrated ethanol. Two ml of the formulation solution were placed in a 6 cc glass vial. The vials were sealed with a Teflon-faced cap and stored for 48 hours at 80° C. They were analyzed by HPLC for the concentration of paclitaxel. The results were as follows. It was found that 98.2% of the paclitaxel remained in the sample treated with charcoal whereas 42.2% of the paclitaxel remained in the untreated sample. Thus, the processes of the present invention and the formulations produced thereby possess greater storage stability, particularly compared to identical formulations made without the treatment or purification of the present invention.

The pH value of both samples was measured following 1:10 dilution with water. The potassium concentration and the color of the samples were analyzed as well. The results are shown in the Table below.

TABLE

| | % Paclitaxel remaining |
|---|---|
| Sample | (80° C. for 48 hours) |
| Charcoal Treated Cremophor EL ® | 98.2 |
| Untreated Cremophor EL ® | 42.2 |

The pH value of the samples was measured following 1:10 dilution with water. The potassium concentration of samples was analyzed. The color of the samples also was measured as shown in following:

| | | | Color (Vis. |
|---|---|---|---|
| Sample | K (ppm) | pH | 425 nm, AU) |
| Charcoal Treated Cremophor EL ® | 137.5 | 4.27 | 0.0381 |
| Untreated Cremophor EL ® | 398.7 | 6.06 | 0.0516 |

As used herein, the term “about” is intended to convey that the numbers and ranges disclosed herein are flexible and that practice of the present invention using temperatures, concentrations, amounts, contents, surface areas, etc. outside of the range or different from a single value will achieve the desired result, namely reducing impurities. The term typically includes a deviation of ±10% of any value it modifies.

### PUBLICATIONS

1. MacEachem-Keith, et al., Anal. Chem. 69:72-77 (1997).
2. Sampedro, et al., J. Microencapsulation 11(3):309-318 (1993).
3. Balasubramanian, et al., Solvent- and Concentration-Dependent Molecular Interactions of Taxol (Paclitaxel), Journal of Pharmaceutical Sciences 83(10) (1994).
4. Merisko-Liversidge, et al., Formulation and Antitumor Activity Evaluation of Nanocrystalline Suspensions of Poorly Soluble Anticancer Drugs, Pharmaceutical Research 13(2)(1996).
5. Sharma, et al., Novel Taxol Formulations: Preparation and Characterization of Taxol-Containing Liposomes, Pharmaceutical Research 11(6) (1994).
6. Adams, et al., J. Natl. Cancer Inst. Monogr. 15,141 (1993).
7. Dordunoo, et al., International Journal of Pharmaceutics 133:191-201 (1996).
8. Tarr, et al., A New Parenteral Vehicle for the Administration of Some Poorly Water Soluble Anti-Cancer Drugs, Journal of Parenteral Science & Technology 41(1) (1987).
9. Kingston, Pharma. Ther. 52:1-34 (1991).
10. Wenk, et al., Paclitaxel Partitioning into Lipid Bilayers, Journal of Pharmaceutical Sciences 85(2) (1996).

### INDUSTRIAL APPLICABILITY

The present invention is useful the preparation of compositions, particularly pharmaceutical compositions containing polyethoxylated castor oils.

All patent and non-patent publications cited in this specification are indicative of the level of skill of those skilled in the art to which this invention pertains. All these publications are herein incorporated by reference to the same extent as if each individual publication or patent application was specifically and individually indicated as being incorporated herein by reference.

1. A polyoxyethylated castor oil produced by:
preparing a suspension of activated charcoal and a polyoxyethylated castor oil wherein the activated charcoal has a median surface area of about 900 m²/g, and is present in the suspension in an amount of from 3 to about 20% (w/w);
heating said suspension at a temperature of from about 30° C. to about 60° C. for a period of time of from about 1 to about 6 hours; and
filtering the heated suspension to separate the activated charcoal from the castor oil.

2. The castor oil of claim 1 which is Cremophor EL®.

3. A formulation comprising the castor oil of claim 1 and an active agent.

4. The composition of claim 3 wherein said active agent is an anti-neoplastic agent.

5. The composition of claim 4 wherein said anti-neoplastic agent is a taxane.

6. The composition of claim 5 wherein said taxane is paclitaxel.

7. The composition of claim 5 further comprising an alcohol.

8. The composition of claim 7 wherein said alcohol comprises dehydrated ethanol.

9. The composition of claim 5 further comprising an acid.

10. The composition of claim 9 wherein said acid is citric acid.

11. A polyoxyethylated castor oil formulation produced by:
preparing a suspension of activated charcoal and a polyoxyethylated castor oil wherein the activated charcoal has a median surface area of about 900 m²/g, and is present in the suspension in an amount of from 3 to about 20% (w/w);
heating said suspension at a temperature of from about 30° C. to about 60° C. for a period of time of from about 1 to about 6 hours;
filtering the heated suspension to separate the activated charcoal from the castor oil; and
adding an active agent to the filtered castor oil.

12. The formulation of claim 11 wherein the active agent is a taxane

13. The formulation of claim 12 wherein the taxane is paclitaxel.

14. The formulation of claim 13 wherein said adding further comprises adding dehydrated ethanol.

15. The formulation of claim 14 wherein said adding further comprises adding citric acid.

16. The formulation of claim 15 wherein said castor oil is Cremophor EL®.

17. A polyoxyethylated castor oil produced by:
preparing a suspension of activated charcoal and a polyoxyethylated castor oil; and
separating the activated charcoal from the polyoxyethylated castor oil.

18. The castor oil of claim 17 wherein the charcoal is present in the suspension in an amount of from 3 to about 20% (w/w).

19. The castor oil of claim 17 wherein the activated charcoal is present in the suspension in an amount of from about 5 to about 10% (w/w).

20. The castor oil of claim 17 wherein the activated charcoal has a surface area ranging from about 500 m²/g to about 1300 m²/g.

21. The castor oil of claim 17 further comprising heating the suspension prior to said separating.

22. The castor oil of claim 21 wherein said heating is conducted at a temperature of from about 30° C. to about 60° C.

23. The castor oil of claim 22 wherein said heating is conducted for a period of time of from about 1 to about 6 hours.

24. The castor oil of claim 17 wherein said separating comprises filtering the suspension.

25. The castor oil of claim 24 wherein said filtering comprises at least two filtration steps using filters with successively smaller apertures.
